(19) United States
(12) Patent Application Publication
(10) Pub. No.: US 2010/0261751 A1
(43) Pub. Date: Oct. 14, 2010

(54) METHOD FOR DETERMINING ONE OF THE TWO HUMAN IMMUNODEFICIENCY VIRUS (HIV) INTEGRASE ENZYMATIC ACTIVITIES

(76) Inventors: Marnix Van Loock, Mechelen (BE); Geert Henri Mersseman, Mechelen (BE)

Correspondence Address: PATTON BOGGS LLP, 8484 WESTPARK DRIVE, SUITE 900, MCLEAN, VA, 22102, US

(21) Appl. No.: 12/742,374

(22) PCT Filed: Dec. 5, 2008
(86) PCT No.: PCT/EP2008/066846

§ 371 Date: May 11, 2010

(30) Foreign Application Priority Data
Dec. 6, 2007 (EP) 07122467.9

(51) Int. Cl. A61K 31/4375; C12Q 1/68; A61K 31/47; C07D 471/04; C07D 215/00; A61P 31/18

(52) U.S. Cl. 514/300; 435/6; 514/312; 546/122; 546/156

(57) ABSTRACT

The invention concerns a method for determining one of the two Human Immunodeficiency Virus (HIV) integrase enzymatic activities, in particular 3′-end processing, in an in vitro assay.

The invention relates to a method for determining one of the two Human Immunodeficiency Virus (HIV) integrase enzymatic activities, in particular 3′-end processing, in an in vitro assay.

Millions and millions of people have been infected with the human immunodeficiency virus, the causative agent of acquired immune deficiency syndrome (“AIDS”), since the early 1980s.

HIV infection in humans is now pandemic. As of January 2006, the Joint United Nations Programme on HIV/AIDS (UNAIDS) and the World Health Organization (WHO) estimate that AIDS has killed more than 25 million people since it was first recognized on Dec. 1, 1981, making it one of the most destructive pandemics in recorded history. In 2005 alone, AIDS claimed an estimated 2.4-3.3 million lives, of which more than 570,000 were children. It is estimated that about 0.6% of the world's living population is infected with HIV. A third of these deaths are occurring in sub-Saharan Africa, retarding economic growth and increasing poverty. According to current estimates, HIV is set to infect 90 million people in Africa, resulting in a minimum estimate of 18 million orphans. Antiretroviral treatment reduces both the mortality and the morbidity of HIV infection, but routine access to antiretroviral medication is not available in all countries.

HIV is different in structure from other retroviruses. It is about 120 nm in diameter (120 billionths of a meter; around 60 times smaller than a red blood cell) and roughly spherical.

It is composed of two copies of positive single-stranded RNA that codes for the virus's nine genes enclosed by a conical capsid composed of 2,000 copies of the viral protein p24. The single-stranded RNA is tightly bound to nucleocapsid proteins, p7 and enzymes needed for the development of the virion such as reverse transcriptase, proteases, ribonuclease and integrase. A matrix composed of the viral protein p17 surrounds the capsid ensuring the integrity of the virion particle. This is, in turn, surrounded by the viral envelope which is composed of two layers of fatty molecules called phospholipids taken from the membrane of a human cell when a newly formed virus particle buds from the cell. Embedded in the viral envelope are proteins from the host cell and about 70 copies of a complex HIV protein that protrudes through the surface of the virus particle. This protein, known as Env, consists of a cap made of three molecules called glycoprotein (gp) 120, and a stem consisting of three gp41 molecules that anchor the structure into the viral envelope. This glycoprotein complex enables the virus to attach to and fuse with target cells to initiate the infectious cycle. Both these surface proteins, especially gp120, have been considered as targets of future treatments or vaccines against HIV.

Of the nine genes that are encoded within the RNA genome, three of these genes, gag, pol, and env, contain information needed to make the structural proteins for new virus particles and the viral enzymes contained in them. Env, for example, codes for a protein called gp160 that is broken down by a viral enzyme to form gp120 and gp41. The six remaining genes, tat, rev, nef, vif, vpr, and vpu (or vpx in the case of HIV-2), are regulatory genes for proteins that control the ability of HIV to infect cells, produce new copies of virus (replicate), or cause disease. The protein encoded by nef, for instance, appears necessary for the virus to replicate efficiently, and the vpu-encoded protein influences the release of new virus particles from infected cells. The ends of each strand of HIV RNA contain an RNA sequence called the long terminal repeat (LTR). Regions in the LTR act as switches to control production of new viruses and can be triggered by proteins from either HIV or the host cell.

HIV integrase itself is a 32 kDa protein produced from the C-terminal portion of the pol gene product. Integrase is an enzyme produced by a retrovirus (including HIV) that enables its genetic material to be integrated into the DNA of the infected cell and is therefore an attractive potential target for new anti-HIV therapeutics.

The HIV integrase protein contains three domains:
an N-terminal HH-CC zinc finger domain (HX₃₋₇HX₂₃₋₃₂CX₂C where H is histidine, C is cysteine and X is any amino acid) believed to be partially responsible for multimerization,
a central catalytic domain containing three absolutely conserved residues that make up the catalytic triad, an aspartic acid residue at position 64, another aspartic acid at position 116, and a glutamic acid at position 152.
a C-terminal domain containing an SH3 fold and believed to be involved in nonspecific DNA binding and tetramerization.

Both the central catalytic domain and C-terminal domains have been shown to bind both viral and cellular DNA. Biochemical data and structural data suggest that integrase functions as a dimer or a tetramer.

Additionally, several host cellular proteins have been shown to interact with integrase and may facilitate the integration process. Integration occurs following production of the double-stranded viral DNA by the viral DNA polymerase, reverse transcriptase. Integrase acts to insert the proviral DNA into the host chromosomal DNA, a step which is essential for HIV replication.

HIV integrase catalyzes two reactions;
3′-end processing, in which two adjacent deoxynucleotides are removed from the 3′ ends of the viral DNA and.
the strand transfer reaction, in which the processed 3′ ends of the viral DNA are covalently ligated to the host chromosomal DNA.

Integration of the proviral DNA is essential for the subsequent transcription of the viral genome which leads to production of new viral genomic RNA and viral proteins needed for the production of the next round of infectious virus. Essentially, integration is a key step in allowing viral DNA to become a permanent member of the host genome. Therefore 3′-end processing and strand transfer, above mentioned, are attractive potential targets for new anti-HIV therapeutics.

Currently, five classes of antiretroviral drugs are used to treat infection by Human Immunodeficiency Virus (HIV), i.e. protease inhibitors (PIs), two classes of reverse transcriptase inhibitors (nucleoside reverse transcriptase inhibitors abbreviated as NRTI and non-nucleoside reverse transcriptase inhibitors abbreviated as NNRTI), and entry inhibitors, sometimes called fusion inhibitors (FIs) and integrase inhibitors (INIs). Several new antiretroviral targets are being explored as potential drug targets. For the time being entry inhibitors and integrase inhibitors are most promising. Integrase inhibitors are a promising new class of antiretrovirals interfering with HIV replication by blocking the ability of the virus to integrate into the genetic material of human cells.

Modern anti-HIV drugs target different stages of the HIV life cycle and a variety of enzymes essential for HIV's replication and/or survival. Amongst the drugs that have so far been approved for AIDS therapy are nucleoside reverse transcriptase inhibitors (“NRTIs”) such as AZT, ddl, ddC, d4T, 3TC, abacavir and tenofovir; non-nucleoside reverse transcriptase inhibitors (“NNRTIs”) such as nevirapine, efavirenz, and delavirdine; protease inhibitors (“PIs”) such as darunavir, saquinavir, ritonavir, indinavir, nelfinavir, amprenavir, lopinavir and atazanavir; and entry inhibitors, such as enfuvirtide and maraviroc. In addition, experiments are currently underway to identify and/or validate anti-HIV drugs that target other HIV polypeptide activities, including, for example, the activities of integrase and RNAse H.

A compound that has been found as so-called first generation INI to be active against HIV integrase is raltegravir (MK-0518) currently, with its analog L870,810,

Nonetheless, in the vast majority of patients none of the antiviral drugs currently approved, either alone or in combination, proves effective either to prevent eventual progression of chronic HIV infection to AIDS or to treat acute AIDS. This phenomenon is due, in part, to the high mutation rate of HIV and the rapid emergence of mutant HIV that are resistant to antiviral therapeutics upon administration of such drugs to infected individuals.

Therefore, the need to screen and thus to identify new structures of HIV integrase inhibitors, so-called second generation INIs, persists.

Traditionally, integrase activities have been measured by low-throughput gel-based assays involving radioactive labeled oligonucleotides. The assay is highly sensitive and commonly used for inhibitor screening. However, the disadvantages of the method are that: (i) it requires radiolabeled substrates, special equipment and appropriate handling of hazardous radioactive waste; and (ii) it is inconvenient to process a large amount of reactions due to the low-throughput format. Compared with the radiolabeled substrate assay, biotin-labeled microtiter plate assays are safe and applicable for high-throughput analysis, and the products of integrase reactions are easy to measure with a spectrophotometer.

However, major drawbacks of these microtiter plate assays are the low dynamic range and intensive labor because of the necessity for plate coating and repeated washing.

On the other hand fluorescence technology, with the advantages of high specificity and sensitivity, has become an important tool for studying nucleic acids and protein/DNA interactions.

Recently, molecular beacons have been widely used in the investigation of DNA-protein interactions including DNA cleavage and ligation reactions because of their high sensitivity, simplicity and real-time monitoring.

As mentioned above although several compounds have been found to be active against HIV integrase, none have completed full clinical trials so far, so there is still a high need to screen and to identify new second generation HIV integrase inhibitors which can stand the mutation rate of HIV accordingly.

The instant disclosure describes a novel in vitro assay to elucidate and/or evaluate new potential HIV integrase inhibitors, but also currently approved and experimental compounds that target HIV integrase.

The present invention concerns a method for determining one of the two Human Immunodeficiency Virus (HIV) integrase enzymatic activities, in particular 3′-end processing, in an in vitro assay by contacting:
a double-stranded nucleic acid corresponding to the long terminal repeat (LTR) end (U5) of HIV-1 of about 20 base pairs comprising SEQ ID NO: 1 and SEQ ID NO: 2 wherein SEQ ID NO:1 comprises a terminal dinucleotide GT having at the 3′ end a fluorophore and wherein SEQ ID NO: 2 is the reverse complement of SEQ ID NO:1 having at the 5′ end a quencher in close proximity to said fluorophore whereby said fluorophore and said quencher are not interfering with the enzymatic function of said HIV integrase and
thereafter determining the 3′-end processing of added HIV integrase by measuring the increase of fluorescence as a consequence of release of dinucleotide GT containing fluorophore from said double-stranded nucleic acid.

In a preferred embodiment SEQ ID NO: 1 comprises an additional three (3) nucleotides CAG or GTC attached at the 5′-end (SEQ ID NO: 3 and SEQ ID NO: 4 respectively).

The fluorophore used in the current invention is, for instance, fluorescein or Alexa 488. The quencher is for instance Dabcyl.

(SEQ ID NO: 1) is 5′-TGTGGAAAATCTCTAGCAGT-3′-
alx488
(SEQ ID NO: 2) is dabcyl-5′-ACTGCTAGAGATTTTCCACA-
3′
(SEQ ID NO: 3) is 5′-CAGTGTGGAAAATCTCTAGCAGT-3′-
alx488
(SEQ ID NO: 4) is 5′-GTCTGTGGAAAATCTCTAGCAGT-3′-
alx488

Part of the invention is also a method above described but further comprising adding a candidate integrase HIV inhibitor compound and determining the change in HIV integrase enzymatic activity, in particular 3′-end processing, as a consequence of said compound addition, relative to the condition lacking said compound.

The compound thus identified can subsequently be formulated in a pharmaceutically acceptable form, by for instance, mixing the compound identified or a derivative or homologue thereof with a pharmaceutically acceptable carrier.

Said identified compound and/or said compound formulated in a pharmaceutical composition can be used to inhibit or prevent HIV integration in a cellular genome.

With “3′-end processing” is meant, the process in which the 3′ proximal dinucleotide is removed from a 3′ end of the viral DNA.

With “fluorophore” is meant, in analogy to a chromophore, a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore.

Fluorescein isothiocyanate, a reactive derivative of fluorescein, has been one of the most common fluorophores chemically attached to other, non-fluorescent molecules to create new and fluorescent molecules for a variety of applications. Other historically common fluorophores are derivatives of rhodamine, coumarin and cyanine.

A new generation of fluorophores such as the Alexa Fluors and the DyLight Fluors are generally more photostable, brighter, and less pH-sensitive than other standard dyes (e.g. fluorescein, rhodamine) of comparable excitation and emission.

The Alexa Fluor family of fluorescent dyes is produced by Molecular Probes, a subsidiary of Invitrogen. Alexa Fluor dyes are typically used as cell and tissue labels in fluorescence microscopy and cell biology.

The excitation and emission spectra of the Alexa Fluor series cover the visible spectrum and extend into the infrared. The individual members of the family are numbered according roughly to their excitation maxima (in nm).

Alexa Fluor dyes are synthesized through sulfonation of coumarin, rhodamine, xanthene (such as fluorescein), and cyanine dyes. Sulfonation makes Alexa Fluor dyes negatively charged and hydrophilic.

Similar alternatives include the Hilyte fluors from AnaSpec and DyLight Fluors from Pierce (Thermo Fisher Scientific).

With “quencher” is meant a non-fluorescent dye that absorbs light but does not emit it. A quencher can be fluorescent though, however for the purpose of the invention described herein the quencher must not inhibit enzymatic activity. It is used in conjunction with regular fluorophores: when they are within range no emissions are detected but when they are separated the fluorophore's emission is detected.

Dabcyl (dimethylaminoazosulphonic acid) absorbs in the green spectrum and is often used with fluorescein. (Dabcyl has a nearly identical absorption but has a sulphonyl chloride to form more stable conjugates, instead of a succinimidyl ester)

### EXAMPLE

The method according to the invention for determining one of the two Human Immunodeficiency Virus (HIV) integrase enzymatic activities, in particular 3′-processing, in an in-vitro assay is hereafter exemplified:
1. thaw 10× buffer (store frozen in 1 to 2 ml aliquots of 250 mM 3-morpholinopropanesulfonic acid (MOPS) pH 7.2 100 mM DTT, 100 mM MgCl2, 150 mM potassium glutamate)
2. thaw stocks of −80° C. stored proteins (integrase, wt, 49 μM in (1M NaCI, 20 mM DTT, 7.5 mM CHAPS, 10% glycerol, 50 mM Tris, pH7.4) and LEDGF, wt, 20 μM in same storage buffer) on ice
3. thaw Q-probe stored at −20° C. in 100 mM NaCl, 10 mM Tris pH 8.0, 5 μM make Q-probe by annealing DNA oligonucleotides 5′-TGTGGAAAATCTCTAGCAGT-3′-a1×488 (SEQ ID NO: 1) to dabcyl-5′-ACTGCTAGAGATTTTCCACA-3′ (SEQ ID NO: 2) in 100 mM NaCl, 10 mM Tris pH 8.0, 5 μM of the alexa488 labeled oligo, 7 μM of the Dabcyl labeled oligo, heating to 95° C. followed by gradual cooling over ˜30′ to RT. The probe is referred to as being at 5 μM.
4. make a master mix of 150 nM Integrase, 100 nM LEDGF, 50 nM Q-probe by dispensing the appropriate volumes of H₂O, 10× buffer, 5 μM Q-probe, LEDGF stock and integrase stock
5. dispense 10 μl 0.5% SDS into columnsl and 2 (negative controls) of a 384 well black bottom plate containing a dilution series of 8 different compounds in the remaining wells
6. dispense 40 μl master mix into all wells
7. incubate 2 hrs at 37° C.
8. dispense 10 μl 0.5% SDS into all wells except columns 1 and 2
9. measure fluorescence, excitation 488 nM, and emission 538 nM

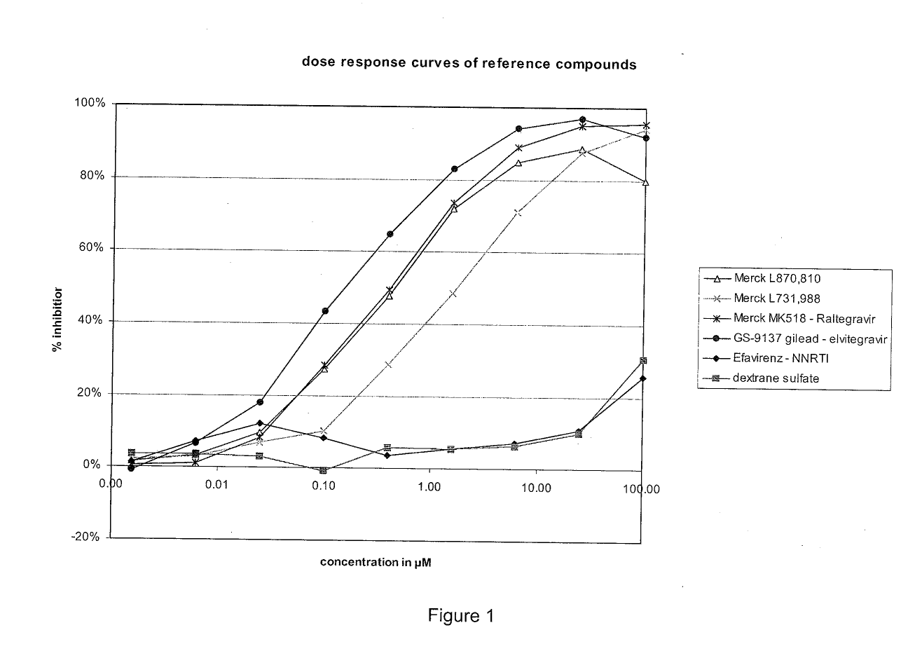

Reference compounds for strand transfer showed reproducible dose response curves. (FIG. 1). The most potent integrase inhibitors like Merck L870,810 naphtyridine or Gilead GS-9137 showed IC50s of around 1 μM, while control compounds like Efavirenz (EFV) did not inhibit.

One of the first diketo acids, Merck L731,988, only showed a weak response (as it does in strand transfer assays). These findings confirm that known catalytic site inhibitors also inhibit 3′-end processing and that the inhibition can be measured with reasonable sensitivity in the so-called 3dQ assay according to the invention.

In summary, a short (˜20 bp) double stranded U5 LTR substrate with a fluorophore on the 3′ end of the GT sequence, and a dark quencher on the 5′ end of complementary DNA strand was constructed. The fluorophore of this construct was nearly completely quenched due to the close proximity of the dark quencher.

As a consequence of 3′-end processing, the fluorophore on the GT dinucleotide was no longer be part of the double strand substrate and therefore, no longer in close proximity to the quencher, resulting in fluorescence. Inhibiting HIV integrase generated a dose response in fluorescence reduction.

1. A method for determining one of the two Human Immunodeficiency Virus (HIV) integrase enzymatic activities, in particular 3′-end processing, in an in vitro assay by contacting:
a double-stranded nucleic acid corresponding to the long terminal repeat (LTR) end (U5) of HIV-1 of about 20 base pairs comprising SEQ ID NO: 1 and SEQ ID NO: 2 wherein SEQ ID NO:1 comprises a terminal dinucleotide GT having at the 3′ end a fluorophore and wherein SEQ ID NO: 2 is the reverse complement of SEQ ID NO:1 having at the 5′ end a quencher in close proximity to said fluorophore whereby said fluorophore and said quencher are not interfering with the enzymatic function of said HIV integrase and
thereafter determining the 3′-end processing of added HIV integrase by measuring the increase of fluorescence as a consequence of release of dinucleotide GT containing fluorophore from said double-stranded nucleic acid.

2. The method according to claim 1 wherein SEQ ID NO: 1 comprises an additional three (3) nucleotides CAG or GTC attached at the 5′-end (SEQ ID NO:3 and SEQ ID NO:4).

3. The method according to claim 1 wherein the fluorophore is fluorescein isothiocyanate (FITC) or Alexa 488 and said quencher is Dabcyl.

4. The method according to claim 1 further comprising adding a candidate integrase HIV inhibitor compound and determining the change in HIV integrase enzymatic activity, in particular 3′-end processing, as a consequence of said compound addition, relative to the condition lacking said compound.

5. The method according to claim 4, further comprising formulating the compound identified in a pharmaceutically acceptable form.

6. A method for the production of a pharmaceutical composition comprising the method of claim 4 and furthermore mixing the compound identified or a derivative or homologue thereof with a pharmaceutically acceptable carrier.

7. Use of a compound as identified by the method of claim 4 or the use of the pharmaceutical composition comprising said compound to inhibit or prevent Human Immunodeficiency Virus (HIV) integration in a cellular genome.


SEQUENCE LISTING (4 sequences)

SEQ ID NO: 1 - DNA, 20 bp, Human immunodeficiency virus
tgtggaaaatctctagcagt

SEQ ID NO: 2 - DNA, 20 bp, Human immunodeficiency virus
actgctagagattttccaca

SEQ ID NO: 3 - DNA, 23 bp, Human immunodeficiency virus
cagtgtggaaaatctctagcagt

SEQ ID NO: 4 - DNA, 23 bp, Human immunodeficiency virus
gtctgtggaaaatctctagcagt